(19) United States
(12) Patent Application Publication
(10) Pub. No.: US 2014/0323575 A1
(43) Pub. Date: Oct. 30, 2014

(54) TYROSINE ISOMERS AS THERAPEUTIC AGENTS

(71) Applicants: CONSEJO NACIONAL DE INVESTIGACIONES CIENTIFICAS Y TECNICAS (CONICET), Buenos Aires (AR); INIS BIOTECH LLC, Milford Kent Country, DE (US)

(72) Inventors: Raul A. Ruggiero, Provincia de Buenos Aires (AR); Juan Bruzzo Iraola, Buenos Aires (AR); Oscar Bustuoabad, Buenos Aires (AR); Paula Chiarella, Buenos Aires (AR); Roberto Meiss, Buenos Aires (AR)

(21) Appl. No.: 14/357,457

(22) PCT Filed: Nov. 9, 2012
(86) PCT No.: PCT/IB2012/056312

§ 371 Date: May 9, 2014

Related U.S. Application Data
(60) Provisional application No. 61/558,833, filed on Nov. 11, 2011.

(51) Int. Cl. A61K 31/198

(52) U.S. Cl. CPC A61K 31/198
USPC 514/567

(57) ABSTRACT

The present invention relates to prevention or treatment of diseases related to abnormal cell proliferation, such as cancer, by administering meta- or ortho-tyrosine to a subject in need thereof, for instance a human subject. More specifically, the present invention provides isomers of tyrosine for its use in the prevention or treatment of a disease and in the preparation of pharmaceutical compositions, methods of treating or preventing diseases, such as cancer and cancer metastases, and pharmaceutical compositions containing meta- and/or ortho-tyrosine.

The present invention relates to prevention or treatment of diseases related to abnormal cell proliferation, such as cancer, by administering meta- or orto-tyorosine to a subject in need thereof, for instance a human subject. More specifically, the present invention provides isomers of tyrosine for its use in the prevention or treatment of a disease and in the preparation of pharmaceutical compositions, methods of for treating or preventing diseases, such as cancer and cancer metastases, and pharmaceutical compositions containing meta- and/or orto-tyorosine.

### BACKGROUND OF THE INVENTION

Concomitant tumor resistance (CR) is a phenomenon in which a tumor-bearing host inhibits or retards the growth of secondary tumor implants. It was first described in 1906 (Ehrlich P. 1906. Experimentelle Carcinomstudien an mausen. Arb Inst Exp Ther Frankfurt 1:77-103), but apart from a few isolated studies (Bashford E., Murray J. and Haaland M. 1908. General results of propagation of malignant newgrowths. In: Bashford E, Editor. Third scientific report on the investigation of the Imperial Cancer Research Fund. Vol. 3: 262-268. Taylor and Francis, London; Woglom W H. 1929. Immunity to transplantable tumors. Cancer Rev 4:129-209), this phenomenon remained virtually forgotten for about 60 years. After a renewal of interest in this concept, some groups studied it primarily by using tumor models in mice, rats, and hamsters (Southam C M. 1964. Host defense mechanisms and human cancer. Ann Inst Pasteur (Paris) 107:585-597; Keller R. 1985. Repression of lymphatic metastasis by a second implant of the same tumor. Invasion Metastasis 5:295-308, Franco M., Bustoabad O. D., di Gianni P. D., Goldman A., Pasqualini C. D., and Ruggiero R. A. 1996. A serum-mediated mechanism for concomitant resistance shared by immunogenic and non-immunogenic murine tumors. Br J Cancer 74: 178-186). However, CR has not received as much attention as other areas of cancer research, despite the fact that it has been detected in association with human cancer and despite its relevance to the mechanisms of metastases control.

Resistance of cancer patients to reinoculation of autologous tumor cells was originally described by Southam (Southam, op. cit.) and Brunschwig and colleagues (Brunschwig A., Southam C. M., and Levin A. G. 1965. Host resistance to cancer. Clinical experiments by homotransplants, autotransplants and admixture of autologous leucocytes. Ann Surg 162: 416-425), who showed that the antitumor resistance to autotransplantation was more profound in patients with localized cancer than in those with regional or distant metastases.

Concerning the relevance of CR for mechanisms of metastasis control, it has been observed that the removal of human and murine tumors may be followed by an abrupt increase in metastatic growth (i.e. Sugarbaker E. V., Thomthwaite J., and Ketcham A. S. 1977. Inhibitory effect of a primary tumor on metastasis. In: Day S. B., Myers W., Stansly P., Garattini S., and Lewis M., eds. Cancer invasion and metastasis. Biological mechanisms and therapy. p. 227-240. Raven, New York; Lange P. H., Hekmar K., Bosi G., Kennedy B. J., and Fraley E. E. 1980. Accelerated growth of testicular cancer after cytoreductive surgery. Cancer 45: 1498-1506; Goerelik E. 1982. Antimetastatic concomitant immunity. In: Liotta L. A. and Hart I. R., eds. Tumor invasion and metastasis. Martinus Nijhoff Publishers, The Hage; Bonfil R. D., Ruggiero R. A., Bustoabad O. D., Meiss R. P., and Pasqualini C. D. 1988. Role of concomitant resistance in the development of murine lung metastases. Int J Cancer 41: 415-422). This suggests that in certain circumstances, the primary tumor exerts a controlling action on its metastases, which can be considered as secondary tumor implants that developed spontaneously during the primary tumor growth.

In the experimental setting, accelerated growth of spontaneous metastases after excision of the primary tumor was observed almost a century ago by Tyzzer (Tyzzer E. E. 1913. Factors in the production and growth of tumor metastases. J Med Res 28: 309-332.1). He observed that although the surgical removal of a primary murine tumor prolonged the survival of mice, the size of the developed metastatic nodules was larger than in mice bearing the primary tumor. In the last 50 years, different groups have studied the growth of spontaneous and experimentally induced metastases in tumor-bearing hosts (i.e. Goerelik, op. cit.; Bonfil, op. cit.; Ketcham, A. S., Kinsey D. L., Wexler H., and Mantel N. 1961. The development of spontaneous metastases after the removal of a “primary” tumor. II. Standardization protocol of 5 animal tumors. Cancer 14: 875-882; Guba M., Cernaianu G., Koehl G., Geissler E. K. et al. 2001. A primary tumor promotes dormancy of solitary tumor cells before inhibiting angiogenesis. Cancer Res 61: 5575-5579). A rather general pattern derived from these experiments can be summarized as follows: The outcome of the removal of a subcutaneous metastatic tumor depended on the size of the local tumor removed. When small tumors were surgically excised, the lungs were left with very few metastatic cells compared with the number in the lungs of tumor-bearing mice in which the primary tumor continued to shed numerous cells into the circulation. As a consequence, the total mass of proliferating metastatic cells in tumor-bearing mice exceeded the growth of the fewer cells existing in the lungs of the tumor-excised mice. At this stage, tumor excision significantly prolonged the survival of the mice. When medium-sized tumors were removed, equilibrium could be reached between the effect of suppression exerted by the primary tumor and the shedding of potentially metastatic cells. Consequently, the total mass of proliferating metastatic cells was similar in both tumor-bearing and tumor-excised mice, because although the tumor-excised mice displayed fewer lung metastatic foci, each focus was of a larger size. At this stage, tumor removal still (albeit modestly) prolonged the survival of the operated mice, presumably because even though both metastatic lung masses were similar, the presence of the primary growing tumor was deleterious for the health of the host. Finally, when large tumors were removed, a higher level of proliferating metastatic cells and larger metastatic nodules compared with those present in tumor-bearing mice were observed. At this stage, tumor excision resulted in a significantly reduced survival rate in the group of operated mice.

In clinical settings, an accelerated growth of metastases following tumor resection has been suspected for decades (Demicheli R., Retsky M. W., Hrushesky W. J. M., Baum M., and Gukas I. D. 2008. The effects of surgery on tumor growth: a century of investigations. Ann Oncol 19: 1821-1828). However, studies comparing metastatic growth in patients with nonexcised tumors (expectant management) with that in patients who have undergone tumor resection (surgical management) are required to definitively show such an effect. Because surgery is one of the primary treatment modalities for solid cancers, such studies are not performed frequently; however, a few can be found in the literature. For example, Iversen and colleagues (Iversen P., Madsen P. O., and Corle, D. K. 1995. Radical prostatectomy versus expectant treatment for early carcinoma of the prostate. Twenty-three year follow-up of a prospective randomized study. Scand J Urol Nephrol Suppl 172: 65-72) found no benefit with radical prostatectomy over expectant management in a 23-year follow-up study of 111 patients with adenocarcinoma of the prostate. Similarly, Demicheli and colleagues (Demicheli R., Valagussa P., and Bonadona G. 2001. Does surgery modify growth kinetics of breast cancer micrometastases? Brit J Cancer 85: 490-492; Retsky M. W., Demicheli R., Hrushesky W. J. M., Baum M., and Gukas I. D. 2008. Dormancy and surgery-driven escape from dormancy help explain some clinical features of breast cancer. APMIS 116: 730-741) examined death-specific hazard rates in a group of patients with breast cancer who had undergone mastectomy alone in comparison with nonoperated patients obtained from an accepted historical database. The group of nonoperated patients (expectant management) exhibited a single peak between the fourth and fifth years in the hazard rate for death. In contrast, a 2-peak hazard was detected in the group of operated patients. The first peak occurred between the third and fourth years after surgery, followed by a second peak in the eighth year. Similar patterns of tumor recurrence after mastectomy were observed by other investigators (Karrison T. G. Ferguson D. J., and Meier P. 1999. Dormancy of mammary carcinoma after mastectomy. J Natl Cancer Inst 91: 80-85), suggesting that the natural history of breast cancer may be adversely affected in some way by removal of the primary tumor.

In many other types of cancer, because of a lack of nonoperated control patients, investigators have been unable to definitively show an enhancement of regional and distant residual tumor growth (i.e., metastases) after primary tumor removal. However, a significant body of evidence that has accumulated over the last 40 years points in that direction. For example, Sugarbaker and colleagues (Subarbaker, op. cit.) reported a clinical case of a 26-year-old male with a melanoma in the scalp. The disease was clinically localized, and evaluation revealed no disseminated metastases. A wide excision and graft were performed, and numerous subcutaneous nodules as well as visceral metastases appeared 6 weeks postoperatively. Lange and colleagues (Lange, op. cit.) reported a study of 8 patients who underwent cytoreductive surgery for testicular cancer. In each case, tumor cytoreductive surgery led to a much faster growth of regional and distant residual disease than would be expected by assuming an uninterrupted, natural growth of residual tumors that were not apparent at the time of surgery. Similar findings in patients with epithelial ovarian cancer (28) led some investigators to urge caution with respect to cytoreductive surgery (Hoskins W. J. 1989. The influence of cytoreductive surgery on progression-free interval and survival in epithelial ovarian cancer. Bailleres clin Obstet Gynaecol 3: 59-71). The above clinical studies, together with similar investigations carried out with patients affected by similar or other malignancies, strongly suggest that sudden acceleration of metastases may be an undesired outcome of surgical removal of many common human malignancies, including primary melanomas and breast, testicular, ovarian, lung, colorectal, and bladder cancers (i.e. Subarbaker, op. cit.; Lange, op. cit.; Beecken W D, Engl T, Jonas D, and Blaheta R A. 2009. Expression of angiogenesis inhibitors in human bladder cancer may explain rapid metastatic progression after radical cystectomy. Int J Mol Med 23:261-266; Demicheli R, Retsky M W, Hrushesky W J M, Baum M. and Gukas I D. op. cit.; Iversen P, Madsen P O, Corle D K. op. cit.; Coffey J C, Wang J H, Smith M J F, Bouchier-Hayes D, Cotter T G, and Redmond H P. 2003. Excisional surgery for cancer cure: therapy at a cost. Lancet Oncol 4:760-768; Maniwa Y, Kanki M, and Okita Y. 2000. Importance of the control of lung recurrence soon after surgery of pulmonary metastases. Am J Surg 179:122-125; Mitsudomi T, Nishioka K, Maruyama R, Saitoh G, Hamatake M, Fukuyama Y, et al 1996. Kinetic analysis of recurrence and survival after potentially curative resection of nonsmall cell lung cancer. J Surg Oncol 63:159-65; Lacy A M, Garcí a-Valdecasas J C, Delgado S, Castells A, TauráP, PiquéJ M, et al. 2002. Laparoscopy-assisted colectomy versus open colectomy for treatment of non-metastatic colon cancer: a randomised trial. Lancet 359:2224-2229; DeLisser H M, Keirns C C, Clinton E A, and Margolis M L. 2009. “The air got to it:” exploring a belief about surgery for lung cancer. J Natl Med Assoc. 101: 765-771). On the other hand, the phenomenon of concomitant enhancement, in which the presence of a primary tumor stimulates the growth of metastases, has also been observed (Janik P, Bertram J S, and Szaniawska B. 1981. Modulation of lung tumor colony formation by a subcutaneously growing tumor. J Natl Cancer Inst 66: 1155-1158; McAllister S S, Gifford A M, Greiner A L, Kelleher S P, Saelzler M P, Ince T A, et al. 2008. Systemic endocrine instigation of indolent tumor growth requires osteopontin. Cell 133: 994-1005). However, in our experience (Bruzzo J, Chiarella P, Meiss R P, Ruggiero R A. 2010. Biphasic effect of a primary tumor on the growth of secondary tumor implants. J Cancer Res Clin Oncol 136: 1605-1615), the magnitude of this stimulatory effect, when present, proved to be rather modest compared with the magnitude of the inhibitory effect produced by CR.

As noted above, most experimental and clinical reports in the literature provide strong evidence that the process of tumor removal adversely alters the fate of minimal residual disease locally (local recurrence) and systemically (metastasis). In fact, local recurrence and especially metastatic growth are far more serious problems than the original tumor because, in most cases, they ultimately prove to be fatal for the patient. Accordingly, there is a need for methods of preventing or reducing the growth of cancer metastases after surgical injuries or stressors which may promote the escape of metastases from dormancy, such as removal of a primary tumor.

Given that the growth of tumor cells reinoculated into animals bearing a primary tumor mimics the situation observed during metastases formation, it appears that an understanding of the mechanisms underlying the phenomenon of CR could provide insight into the mechanisms that inhibit the growth of metastatic cells in the presence of a primary tumor. This in turn would aid in the design of new strategies to limit the development of metastases that arise after resection of primary tumors.

Different hypotheses have been proposed to explain the phenomenon of CR. According to the immunological hypothesis, the growth of a tumor generates a specific antitumor immune response that may not be strong enough to inhibit the primary tumor growth but will still be able to prevent the development of a relatively small secondary tumor inoculum. This explanation is not very different from that of conventional immunologic rejection of allogeneic tumors in naive mice or immunogenic syngeneic tumors in previously immunized animals. The immunological hypothesis was originally proposed in 1908 by Bashford and colleagues (op. cit.), who also coined the term “concomitant immunity,” by which this phenomenon was known in the past. This interpretation is supported by solid evidence mainly based on experiments with strongly immunogenic murine tumors induced by chemical agents or viruses (Franco et al. op. cit.; North R J. 1984. The murine antitumor immune response and its therapeutic manipulation. Adv. Immuno 135: 89-155). However, it does not provide a satisfactory explanation for the fact that CR has also been observed in association with spontaneous murine tumors of nondetectable immunogenicity (Ruggiero R A, Bustuoabad O D, Bonfil R D, Meiss R P, and Pasqualini C D. 1985. “Concomitant immunity” in murine tumours of non-detectable immunogenicity. Br J Cancer 51: 37-48; Ruggiero R A, Bustuoabad O D, Cramer P, Bonfil R D, and Pasqualini C D. 1990. Correlation between seric antitumor activity and concomitant resistance in mice bearing nonimmunogenic tumors. Cancer Res 50: 7159-7165).

Other investigators (Goerlik, op. cit., Ruggiero et al. 1990, op. cit.; DeWys W D. 1972. Studies correlating the growth rate of a tumor and its metastases and providing evidence for tumor-related systemic growth-retarding factors. Cancer Res 32: 374-379; O'Reilly M S, Holmgren L, Shing Y, Chen C, Rosenthal R A, Moses M, et al. 1994. Angiostatin: a novel angiogenesis inhibitor that mediates the suppression of metastases by a Lewis lung carcinoma. Cell 79: 315-28; Bustuoabad O D, Genovese J A, and Pasqualini C D. 1984. Abrogation of concomitant tumor immunity in mice. Com 8io 2:423-430) have postulated that tumor cells of the primary tumor produce (or induce the production of) antiproliferative nonspecific substances or antiangiogenic molecules that suppress or limit, directly or indirectly, the replication of tumor cells of the second inoculum.

These nonimmunological hypotheses can offer a putative explanation for the phenomenon of CR induced by nonimmunogenic tumors but not for the specific inhibition of secondary tumor implants observed during the growth of immunogenic tumors.

Previous studies on the phenomenon of CR associated with the growth of 17 murine tumors with widely different degrees of immunogenicity resulted in the description of 2 temporally separate peaks of CR during primary tumor growth, which may explain many apparently contradictory results reported by different authors (Woglom, op. cit.; Franco, op. cit.; Goerlik, op. cit.; North, op. cit.). These differences seem to be related to the different stages of tumor growth at which these authors looked for CR, as well as to the different characteristics of both peaks. In effect, the first peak was observed when the primary tumor was small (<500 mm³). It was tumor-specific and thymus-dependent, as it was exhibited in euthymic but not in nude mice, and its intensity was proportional to tumor immunogenicity. A typical immunological rejection, associated with extensive necrosis and a profuse infiltration with polymorphonuclear granulocytes and mononuclear cells, was observed histologically at the site of the second tumor implant undergoing CR. Furthermore, the kinetics of appearance and disappearance of the first peak of CR paralleled the kinetics of appearance and disappearance of cytotoxic antibodies and cell-mediated cytotoxicity against the tumor.

On the other hand, the second peak of CR was induced by both immunogenic and nonimmunogenic large tumors (≧2,000 mm³). It was not tumor-specific or thymus-dependent, as it was exhibited in both euthymic and nude mice, and it did not correlate with tumor immunogenicity. Inhibition of the secondary tumor in the presence of a large primary tumor was not associated with a massive or focal necrosis, or with any host cell infiltration, but it was associated with the presence of noninfiltrating tumor cells (i.e., dormant tumor) located at the inoculation site between the skin and the muscular layer.

Some years ago, an intermediate peak of CR was reported to be associated with a particular type of mid-sized tumors (1,000-1,500 mm³) that restrain secondary tumors indirectly by limiting tumor neovascularization (O'Reilly, op. cit.).

The inhibitory activity of the second peak was partially characterized and indicated a heat-, acid-, and alkali-resistant factor of low molecular weight that apparently was unrelated to other well-characterized, growth-inhibitory molecules (e.g., interferons, TNF-a, TGF-b, angiostatin, and endostatin), taking into account the larger molecular weight of the latter and other physical and biological properties (Di Gianni P D, Franco M, Meiss R P, Vanzulli S, Piazzon 1, Pasqualini C D, et al. 1999. Inhibition of metastases by a serum factor associated to concomitant resistance induced by unrelated murine tumors. Oncol Rep 6: 1073-1084; Ruggiero et al. 1990, op. cit.; Franco M, Bustuoabad O D, Di Gianni P D, Meiss R P, Vanzulli S, Buggiano V, et al. 2000. Two different types of concomitant resistance induced by murine tumors: morphological aspects and intrinsic mechanisms. Oncol Rep 7:1053-1063).

However, despite these efforts, the origin and chemical nature of that factor remained elusive for years. In addition, the question of how such a factor could inhibit the proliferation of a secondary tumor but not that of a large primary one composed of the same type of cells remained unresolved.

### BRIEF DESCRIPTION OF THE INVENTION

The inventors have now isolated and identified meta- and ortho-tyrosine as the serum factor(s) associated with the phenomenon of CR. The inventors also proved its biological antitumor activity in both primary tumors and metastases, and proposed the mechanisms by which these factors effect tumor inhibition.

Accordingly, it is a first object of the present invention an isomer of tyrosine selected from the group consisting of meta- and ortho-tyrosine for its use in preventing or treating a disease, for instance a cancer such as leukemia, fibrosarcoma, primary melanomas and pancreas, breast, testicular, ovarian, lung, colorectal, and bladder cancers. The tyrosine isomers can be used according to the invention for treating primary tumors as well as its metastases.

In another aspect, the invention comprises the use of an isomer of tyrosine selected from the group consisting of meta- and/or ortho-tyrosine in the manufacture of a pharmaceutical composition for preventing or treating a disease.

In another aspect, the invention comprises a method for preventing or treating a disease (such as cancer), wherein meta- and/or ortho-tyrosine are administered in a therapeutically effective amount to a subject in need thereof.

Finally, in yet another aspect, the invention comprises a pharmaceutical composition which comprises an isomer of tyrosine selected from the group consisting of meta- and/or ortho-tyrosine, preferably meta-tyrosine, and a pharmaceutically acceptable carrier. In a particular embodiment, the pharmaceutical composition is for treating cancer, such as a cancer selected from the group consisting of leukemia (including LB leukemia), fibrosarcoma (including MC-C), primary melanomas, carcinomas (including CEI), and breast, testicular, ovarian, lung, colorectal, and bladder cancers. In a more particular embodiment, the pharmaceutical composition is for preventing or reducing the growth of a primary tumor. Alternatively, or in combination with the treatment of a primary tumor, the pharmaceutical composition is for preventing or reducing the growth of cancer metastases after the removal of a primary tumor, or after other surgical injuries or stressors that may promote the escape of metastases from dormancy, in particular, for cancer types known to show sudden acceleration of metastases after surgical removal of a tumor.

### DESCRIPTION OF THE FIGURES

FIG. 1 illustrates the scheme of the purification of the anti-tumor activity associated with the phenomenon of concomitant tumor resistance (CR) starting from serum from mice bearing a subcutaneous LB tumor (size≧2,000 mm³) as described in Example 2 and exhibiting the second peak of CR.

FIG. 2A illustrates a representative experiment showing the HPLC elution profile of the growth-inhibitory activity present in serum from LB tumor-bearing mice, using a gradient of TFA and acetonitrile (first HPLC). FIG. 2B shows the effect of each fraction on in vitro LB tumor cell proliferation ([³H]-thymidine uptake). Number of assays (n)=3 for each fraction and n=6 for control. a, P<0.001 versus control and the other fractions. Serum from normal mice did not exhibit any fraction with growth-inhibitory activity.

FIG. 3A illustrates a representative experiment showing the second HPLC elution profile of the active fraction obtained from the first HPLC, with a gradient of TFA and methanol. Commercial tyrosine, meta-tyrosine (m-Tyr), and ortho-tyrosine (o-Tyr, 60 μg/mL) were comparatively analyzed. FIG. 3B is a high resolution ion-electrospray MS and MS/MS of the 3 peaks obtained from the second HPLC and of commercial tyrosine, meta-tyrosine, and ortho-tyrosine. FIG. 3C is a comparative analysis by MS/MS of the active fraction from the first HPLC and of a mixture composed by commercial tyrosine, meta-tyrosine, and ortho-tyrosine at different relative concentrations.

FIG. 4 illustrates a co-elution experiment in which graded concentrations of m-tyrosine (40 or 80 μg/ml), o-tyrosine (40 or 80 μg/ml) or both (40 or 80 μg/ml of m-tyrosine plus 40 or 80 μg/ml of o-tyrosine) were added into the anti-tumor biological serum sample associated with CR obtained from the first HPLC (acetonitrile in TFA) and applied to the second HPLC (methanol in TFA). Note that only the second peak from the second HPLC is progressively increased with the addition of 40 or 80 μg/ml of m-tyrosine. In the same way, only the third peak is progressively increased with the addition of 40 or 80 μg/ml of o-tyrosine. Finally both, the second and the third peaks are increased with the addition of m-tyrosine plus o-tyrosine. Controls with tyrosine alone, m-tyrosine alone and o-tyrosine alone (200 μg/ml in each case) were coincident with the first, second and third peaks of the sample, respectively, but, for clarity, were not added to the figure. Abscissa: Elution time (min). Ordinate: Absorbance at 274 nm.

FIG. 5A shows the comparative effect of the 3 peaks obtained from the second HPLC and commercial tyrosine, meta-tyrosine (m-Tyr), and ortho-tyrosine (o-Tyr) on in vitro tumor cell proliferation. Each point (percentage of cpm incorporated by LB cells as compared with the control) represents the mean±SE of 3 experiments. Significance versus control, meta-tyrosine and peak 2: P<0.001 at >2.75 μg/mL; peak 2: P<0.05 at 2.75 μg/mL. ortho-tyrosine P<0.001 at 500 to 125 μg/mL; P<0.002 at 30 μg/mL. Peak 3: P<0.002 and P<0.01, at 31 and 15.5 μg/mL, respectively. Rescue of in vitro LB tumor inhibition mediated by meta-tyrosine (150 μg/mL)(B) or ortho-tyrosine (150 μg/mL) (C) by co-culturing with 150 μg/mL of individual protein amino acids. Each value represents the mean±SE of 8 to 18 determinations for (B) and 4 to 9 determinations for (C). a, P<0.01; c, P<0.001 versus meta- or ortho-tyrosine. Data with saline was similar to data obtained with each amino acid alone.

FIG. 6A illustrates the counteracting effect of phenylalanine on the in vivo tumor growth inhibition induced by CR and inhibitory effect of meta-tyrosine (m-Tyr) on in vivo tumor growth mimicking the inhibition induced by CR. Twenty mice received an s.c. implant of 1×10⁶ LB cells in the right flank, and, 7 days later, an s.c. secondary implant of 1×10⁵ LB tumor cells in the left flank. Ten mice did not receive any additional treatment and the secondary implant did not grow [CR: (-▪-)]. The remaining 10 mice received 0.2 mL of phenylalanine (500 μg/mL) at the site of the secondary implant, daily, for 8 days, starting 1 hour after the secondary inoculum [CR+Phe (-□-)]. Controls were 10 mice receiving 1×10⁵ LB tumor cells in the left flank only [control (-Δ-)]. A fourth group received the tumor inoculum in the left flank but also meta-tyrosine at the site of tumor implant (0.2 mL of 500 μg/mL, n=10) or by the intraperitoneal route (0.5 mL of 1,000 μg/mL, n=6), daily, for 8 days, starting 1 hour after tumor inoculum [control+m-Tyr: (-▴-)]. A fifth group received the tumor inoculum in the left flank and also meta-tyrosine (0.2 mL of 500 μg/mL, n=5) at the site of tumor growth, daily for 5 days, starting (see arrow) when LB was an established tumor measuring 60 to 70 mm³ [est. LB+m-Tyr: (-X-)]. Abscissa, days after tumor implant in the left flank. Ordinate, volume of the tumors implanted in the left flank. CR and control+m-Tyr versus control: P<0.001 at day 9 and P<0.002 at days 5 and 7; CR and control+m-Tyr versus CR+Phe: P<0.001 at days 5, 7 and 7; est. LB+m-Tyr versus control and CR+Phe: P<0.002 at day 9. Control+tyrosine or saline, control; CR+tyrosine or saline, CR. FIG. 6B is a microscopic view showing many tumor cells expressing Ki-67 protein in both CR+phenylalanine (B1) and control (B2) groups and few tumor cells expressing Ki-67 in both CR (B3) and control+meta-tyrosine (B4) groups (H×400). FIG. 6C illustrates a representative experiment showing the frequency distribution histogram of viable LB cells from a control tumor (C1), from a secondary tumor inhibited by CR (C2), and from a tumor inhibited by periodic intraumoral injection of meta-tyrosine (C3). Samples of tumor cells were collected 9 days after the s.c. inoculation of 1×10⁵ LB tumor cells.

FIG. 7 shows the inhibition of spontaneous C7HI-lung metastases by meta-tyrosine. FIG. 7A illustrates an experiment in which mice bearing a C7HI tumor for 50 days received, between days 50 and 64, a daily intravenous injection of meta-tyrosine (0.3 mL of 1,000 μg/mL) or saline. Mice were sacrificed at day 50 (n=8, before treatment, primary tumor volume 241±16 mm³) or at day 65 for treated (n=12, primary tumor volume 1,080±101 mm³) or control (n=12, primary tumor volume 1,091±88 mm³) mice, and number and size of macroscopic metastases were determined; d=diameter of metastases in mm; a, P<0.05 (Mann-Whitney U test and Student t test) versus control. The difference between the size of the primary tumors at day 65, in experimental and controls was not significant. FIG. 7B depicts a macroscopic (B1; 10×) and microscopic (B3; H&E 100×) view of a lung from an m-tyrosine-treated mouse showing less metastatic foci (arrows) as compared with a control (B2 and B4). H&E, hematoxylin and eosin.

FIG. 8 illustrates the effect of meta-tyrosine on the expression of phosphorylated (p)-Erk 1/2 and p-STAT3 in LB tumor cells using Western blot: 3×10⁵ LB tumor cells in 0.1 mL of medium were cultured in the presence of 0.1 mL of meta-tyrosine (m-Tyr; 250 μg/mL), phenylalanine (Phe, 250 μg/mL), meta-tyrosine (250 μg/mL)+phenylalanine (250 μg/mL), or saline for 3 minutes (to measure p-ERK 1/2) or for 8 hours (to measure p-STAT3). Positive control was obtained by treating LB cells with pervanadate. Controls with acting, total ERK 1/2, and total STAT3 were also added. FIG. 8A illustrates a representative experiment showing p-ERK 1/2 expression. FIG. 8B is a histogram showing the mean of 5 experiments; a, P<0.01 versus saline and versus meta-tyrosine+phenylalanine; P<0.02 versus phenylalanine. FIG. 8C illustrates a representative experiment showing p-STAT3 expression. FIG. 8D is a histogram showing the mean of 6 experiments; a, P<0.01 versus saline and versus phenylalanine; P<0.02 versus meta-tyrosine+phenylalanine. FIG. 8E is the full blot of the experiments of FIGS. 7A (A) and 7C (B).

FIG. 9 shows the inhibition of spontaneous C7HI-lung metastases by meta-tyrosine in an experiment similar to the one of FIG. 7. Mice bearing a C7HI tumor for 50 days received, between days 50 and 71, a daily intravenous injection of 1.0 mg of meta-tyrosine diluted in 0.1 ml of saline, or saline only. Mice were sacrificed ad day 50 (n=12, before treatment) or at day 71 for treated (n=12) or control (n=12) mice, and number and size of macroscopic metastases were determined; d=diameter of metastases in mm. The difference between the size of the primary tumors at day 71, in experimental and controls was not significant.

### DETAILED DESCRIPTION OF THE INVENTION

Tyrosine (4-hydroxyphenylalanine) is one of 21 amino acids known to form proteins in eucariots. In addition to tyrosine, or L-tyrosine, which is the para-isomer in the main arene substitution pattern of the hydroxyphenylalanine, the other 2 isomers of the hydroxyphenylalanine in the ortho-meta-para-substitution pattern, namely meta-tyrosine (m-tyr or 3-hydroxyphenylalanine or L-m-tyrosine) and ortho-tyrosine (o-tyr or 2-hydroxyphenylalanine), also occur naturally, although both are much less abundant than tyrosine and are not known to normally be part of proteins.

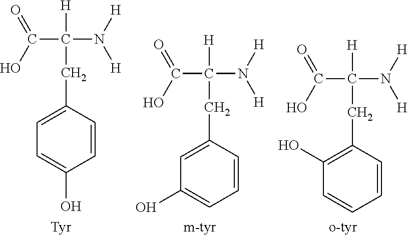

In the present invention, meta-tyrosine and ortho-tyrosine are used in preventing or reducing the growth of cancer metastases after the removal of a primary tumor, or after other surgical injuries or stressors that may promote the escape of metastases from dormancy in a subject in need thereof.

No previous studies have previously reported anti-proliferative effects mediated by m- and o-tyrosine. Gurer-Orhan and collaborators (H. Gurer-Orhan, N. Ercal, S. Mare, S. Pennathur, H. Orhan, and J. W. Heinecke. 2006. Misincorporation of free m-tyrosine into cellular proteins: a potential cytotoxic mechanism for oxidized amino acids. Biochem. J 395: 277-284) while studying alternative mechanisms for oxidative stress and tissue injury during aging and disease, showed that free m-tyrosine and o-tyrosine were toxic to Chinese-hamster ovary (CHO) cells when these cells were incubated in vitro with m- or o-tyrosine for 7-10 days. In that study, 70% of the CHO cells treated with meta-tyrosine were alive 10 days after start of the in vitro culture with meta-tyrosine. This mild cytotoxic effect in the long term is clearly different from the cytostatic effect in the short term (8-18 hs) of meta- and ortho-tyrosine now discovered by the inventors. In the same way, Bertin and collaborators (C. Bertin, L. A. Weston, T. Huang, G. Jander, T. Owens, J. Meinwald, and F. C. Schroeder. 2007. Grass roots chemistry: meta-tyrosine, an herbicidal nonprotein Amino acid. Proc. Natl. Acad. Sci. USA 104: 16964-16969), while studying the development of more environmentally friendly weed management systems, demonstrated that the unusual ability of many fine fescue grasses to outcompete or displace other neighboring plants was based on the phytotoxic properties of their root exudates and that more than 80% of the active fraction was m-tyrosine. Both authors hypothesized that one potential cytotoxicity mechanism could involve mischarging of tRNA and consequent misincorporation of these unnatural isomers of tyrosine into cellular proteins based on their structural similarities with phenylalanine or tyrosine. In turn, this misincorporation could cause structural disruption in proteins or could interfere with the functions of key enzymes such as DNA polymerase which might lead to errors in DNA replication and long-term consequences such as impaired cellular viability.

According to a first aspect, it is an object of the present invention an isomer of tyrosine selected from the group consisting of meta- and ortho-tyrosine for its use in preventing or treating a disease, such as cancer. Since the second peak of CR has been found in many different types of cancer, the inhibitory effect of meta- and ortho-tyrosine is not limited to a particular kind of cancer or a few related kinds of tumors but these isomers can be used in the prevention or treatment of virtually any kind of cancer. However, according to previous research and to the inventor's own experimental data, meta- and ortho-tyrosine are advantageously used for treating some kinds of cancer. For that reason, in a particular embodiment the invention relates to an isomer of tyrosine selected from the group consisting of meta- and ortho-tyrosine for its use in preventing or treating cancer, where the cancer is selected from the group consisting of leukemia (including LB leukemia), fibrosarcoma (including MC-C), primary melanomas, carcinomas (including CEI), and breast, testicular, ovarian, lung, colorectal, and bladder cancers.

By understanding the mechanism by which primary tumors are responsible for the CR phenomenon but are not self-inhibited, the inventors have surprisingly found that, although the CR phenomenon has been exclusively in association to the development of metastasis or implanted secondary tumors, of meta- and ortho-tyrosine can be used also for treatment of primary cancer tumors. Thus, in a particular embodiment it is an object of the present invention an isomer of tyrosine selected from the group consisting of meta- and ortho-tyrosine for its use in preventing or reducing the growth of a primary tumor.

According to another particular embodiment, the invention has for an object an isomer of tyrosine selected from the group consisting of meta- and ortho-tyrosine for its use in preventing or reducing the growth of cancer metastases after the removal of a primary tumor, or after other surgical injuries or stressors that may promote the escape of metastases from dormancy. In particular, the isomers of tyrosine are advantageously used in preventing or reducing the growth of metastases of those types of cancer known to show sudden acceleration of metastases after surgical removal of a tumor, such as primary melanomas and breast, testicular, ovarian, lung, colorectal, and bladder cancers.

Since meta-tyrosine inhibitory effect on in vitro and in vivo proliferation of tumor cells is stronger than that of ortho-tyrosine, a preferred embodiment of the invention consists of meta-tyrosine for its use in preventing or treating a disease, such as cancer, according to any of the uses described above.

Meta-tyrosine and ortho-tyrosine and methods for its preparation are well-known in the art, and both isomers are readily available from commercial suppliers (i.e. Sigma). As an example, a method for the synthesis of ortho-tyrosine was already described in 1956 (Shaw, K., McMillan, A. and Armstrong, M. 1956. Synthesis of o-tyrosine and related phenolic acids. J. Org. Chem. 21 (6): 601-604. A method for the efficient synthesis of meta-tyrosine is described in Bender, D. and Williams, R. 1997. An Efficient Synthesis of (S)-m-Tyrosine. J. Org. Chem. 62(19): 6448:6449.

According to the invention, the tyrosine isomers can be administered in the form of a pharmaceutical composition. Thus, according to another aspect, the present invention comprises the use of an isomer of tyrosine selected from the group consisting of meta- and/or ortho-tyrosine, and preferably meta-tyrosine, in the manufacture of a pharmaceutical composition, such as for preventing or treating cancer, preferably selected from the group consisting of leukemia (including LB leukemia), fibrosarcoma (including MC-C), primary melanomas, carcinomas (including CEI), and breast, testicular, ovarian, lung, colorectal, and bladder cancers.

In a particular embodiment, the pharmaceutical composition is for its use in preventing or reducing the growth of a primary tumor. In another embodiment, the pharmaceutical composition is for its use in preventing or reducing the growth of cancer metastases after the removal of a primary tumor, or after other surgical injuries or stressors that may promote the escape of metastases from dormancy, preferably for preventing or reducing the growth of metastases of a cancer known to show sudden acceleration of metastases after surgical removal of a tumor, such as primary melanomas and breast, testicular, ovarian, lung, colorectal, and bladder cancers.

Within the scope of the present invention, suitable pharmaceutical compositions may comprise a single compound, or mixtures of compounds thereof, comprising meta-tyrosine and/or ortho-tyrosine and a pharmaceutically acceptable diluent, extender, excipient, filler, manufacturing aid, solvent or carrier (collectively referred to herein as a pharmaceutically acceptable carrier). The precise kind of carrier and excipient will depend mostly of the administration route of choice, and has to be consistent with conventional pharmaceutical practices. In a preferred embodiment, the pharmaceutical composition manufactured is a pharmaceutical composition suitable for parenteral, preferably intravenous administration. Complete and detailed reference about formulations and pharmaceutical compositions can be found in Remington: The Science and Practice of Pharmacy; by A. R. Gelmaro (ed.) 20th edition: Dec. 15, 2000, Lippincott, Williams & Wilkins.

According to another aspect, the present invention comprises a method for preventing or treating a disease, such as cancer, by administering a therapeutically effective amount of meta- and/or ortho-tyrosine, and preferably meta-tyrosine, to a subject in need thereof. This method is particularly useful for prevention or treatment of leukemia (including LB leukemia), fibrosarcoma (including MC-C), primary melanomas, carcinomas (including CEI), and breast, testicular, ovarian, lung, colorectal, and bladder cancers.

The method of treatment of the invention can be applied to the treatment of primary tumors, or for reducing the growth of cancer metastases after the removal of a primary tumor, or after other surgical injuries or stressors that may promote the escape of metastases from dormancy in a subject in need thereof. In the latter case, the method is particularly useful in preventing or reducing the growth of metastases of those types of cancer known to show sudden acceleration of metastases after surgical removal of a tumor, such as primary melanomas and breast, testicular, ovarian, lung, colorectal, and bladder cancers.

For preventing or reducing the growth of cancer metastases, the tyrosine isomers or pharmaceutical compositions containing thereof can be administered by oral, nasal, enteral, dermal, intravenous, intracardiac, intramuscular, intraosseous, intraperitoneal, subcutaneous, intrapleural, topical, intradermal, intrauterine, rectal, vaginal, intratumor, intra or periocular, parenteral, vaginal, rectal or intrasynovial routes.

In the method of the invention, advantageously the isomers of tyrosine are administered by injection, including intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospnial and intrasternal injection and infusion. In a preferred embodiment, the isomer of tyrosine is administered intravenously, and more preferably by intravenous injection.

According to a particular embodiment, in the method of the invention the isomer of tyrosine is injected at or near the site of the cancer cells whose growth prevention or reduction is sought.

Pharmaceutical compositions suitable for parenteral administration are well-known in the art, and include sterile aqueous preparations of the active compound such as solutions and suspensions. These aqueous preparations are preferably isotonic with the blood of the recipient, and may contain, in addition to the active compound, distilled water, a solution of a sugar like dextrose in distilled water, saline, Ringer's lactate or Ringer's acetate. The pharmaceutical compositions can also be provided in the form of solids or as concentrated solutions containing the active compound which are diluted with a suitable solvent in the necessary amount as to give a solution or suspension suitable for parenteral administration.

The isomers of tyrosine used in the invention are not toxic or show other adverse effects when administered at doses as high as 33.3 mg/kg of body weight. On the other hand, doses as low as 3.3 mg/kg of body weight are effective in inhibiting the growth of tumors. Accordingly, in a particular embodiment, the method of the present invention comprises administering a therapeutically effective amount of a meta- or ortho-tyrosine isomer to a patient in need thereof, wherein the therapeutically effective amount is between 33.3 mg/kg of body weight and 3.3 mg/kg of body weight, preferably 3.3 mg of meta-tyrosine/kg of the body weight. Assuming that an adult human being weights on average 70 kg, this represents between 23 gr. and 233 gr. of tyrosine isomer per day, and preferably 23 gr. of meta-tyrosine per day for treating a human subject.

According to yet another aspect, the present invention comprises a pharmaceutical composition, such as a pharmaceutical composition for the prevention or treatment of cancer, which comprises an isomer of tyrosine selected from the group consisting of meta- and/or ortho-tyrosine and a pharmaceutically acceptable carrier. Preferably, the isomer of tyrosine is meta-tyrosine.

The several aspects of the present invention will be better understood through the following examples. The scope of the invention, however, is not limited to the examples, as the purpose of these is not to limit the invention but to illustrate particular embodiments of the invention from which alternative embodiments, still within the scope of the invention, will be evident for those skilled in the art.

### Example 1

### Origin of the Factor Associated with CR

LB tumor cells cultures were conditioned with serum coming from normal, splenectomized, adrenalectomized and macrophage depleted tumor bearing Balb/c and Nude mice, and tumor cell proliferation was measured.

Nude, splenectomized, adrenalectomized, and macrophage-depleted tumor-bearing mice exhibited a similar level of serum inhibitory activity in vitro as compared with control tumor-bearing mice (not shown). This suggested that neither tumor cells per se, thymus, spleen, adrenals, nor macrophages play a main role in the generation of the factor. On the contrary, because LB tumor growth is accompanied by overt manifestations of systemic inflammation—evidenced by a significant increase of circulating proinflammatory cytokines (TNF-α and interleukins 1β and 6), phase acute proteins (SAA protein), neutrophils, and myeloid-derived suppressor cells (MDSC; not shown), the relationship between these manifestations and the serum antitumor activity was investigated. A significant reduction of that serum activity was observed upon treatment of LB tumor-bearing mice with different steroidal and nonsteroidal anti-inflammatory agents [(dexamethasone, indomethacin, promethazine, chlorpromazine, phenidone, and nordihydroguaiaretic acid (NDGA)], with gemcitabine [that in the concentration used by us and others (Le H K, Graham L, Cha E. Morales J K, Manjiii M H, and Bear H D. 2009. Gemcitabine directly inhibits myeloid derived suppressor cells in BALB/c mice bearing 4TI mammary carcinoma and augments expression of T cells from tumor-bearing mice. Int Immunopharmacology 9:900-909) sharply depleted the number of MDSCs without lowering the number of T cells, B cells, and macrophages] and catalase (that prevents oxidative damage). L-NAME, that prevents peroxynitrite generation, and losartan, which exhibits a weak anti-inflammatory effect (Krämer C, Sunkornat J, Witte J, Luchtefeld M, Walden M. Schmidt B, et al. 2002. Angiotensin II receptor-independent antiinflammatory and antiaggregatory properties of losartan: role of the active metabolite EXP3179. Circ Res 90:770-776), did not reduce the serum inhibitory activity. The strongest effects were obtained with gemcitabine and catalase (Table 1). Furthermore, we observed that MDSCs isolated from blood from LB tumor-bearing mice exhibiting the second peak of CR—but not from normal mice or from mice bearing a small LB tumor that did not yet induce CR—spontaneously produced a brightly fluorescent FL-1 product of dihydrorhodamine 123 indicating a high production of reactive oxygen species (ROS) by MDSCs, which, in turn, could oxidize phenylalanine to produce meta- and ortho-tyrosine. These data suggested that ROS released by MDSCs are involved in the generation of the serum antitumor factor.

TABLE 1
Effect of gemcitabine, antireactive oxygen, anti-inflammatory,
and antireactive nitrogen species drugs on the inhibitory activity
present in serum from LB tumor-bearing mice

| Serum from | GIU₅₀/mL (X ± SE)^{a} | n^{b} |
|---|---|---|
| Normal mice | 37 ± 7 | 7 |
| LB tumor-bearing mice | 303 ± 30 | 7 |
| LB tumor-bearing mice treated | 40 ± 6 | 2 |
| with Gemcitabine^{c} | | |
| Catalase^{d} | 49 ± 5 | 3 |
| Dexamethasone^{e} | 70 ± 18 | 3 |
| Promethazine^{f} | 80 ± 9 | 3 |
| INDO + NDGA^{g} | 81 ± 16 | 5 |
| Chlorpromazine^{h} | 84 ± 19 | 3 |
| Phenidoneⁱ | 103 ± 19 | 2 |
| NDGA^{j} | 116 ± 14 | 5 |
| INDO^{k} | 142 ± 16 | 6 |
| L-NAME^{l} | 266 ± 34 | 3 |
| Losartant^{m} | 300 ± 30 | 3 |

NOTE:
Recovery of the titer of serum inhibitory activity occurred 72 hours after the last dose of the different agents suggesting that this serum activity is continuously produced in tumor-bearing mice. No effect was observed in serum from normal mice that received the same schedule of treatment.
^{a}GIU₅₀/mL, titer of growth-inhibitory activity was defined as the reciprocal of the serum dilution producing 50% inhibitory of [³H] thymidine uptake by LB tumor cells as compared with medium only and expressed by milliliter of serum.
^{b}n, Number of independentexperiments,
^{c-m}Mice bearing an s.c. LB tumor (volume > 2,000 mm³) received indomethacin (0.5 mg/kg), dexamethasone (0.75 mg/kg), losartan (1 mg/kg), promethazine (1 mg/kg), chiorpromazine (1.2 mg/kg), NDGA (5 mg/kg), phenidone (5 mg/kg), a mixture of indomethacin and NDGA, catalase (350,000 units/kg), L-NAME (25 mg/kg), or gemcitabine (120 mg/kg) by the intraperitoneal route, 48 and 24 hours before testing the titer of serum growth inhibitory activity.
^{c}P: not significant versus normal serum, P < 0.01 versus LB serum,
^{d}P: not significant versus normal serum, P < 0.001 versus LB serum,
^{e}P: not significant versus normal serum, P < 0.002 versus LB serum.
^{f}P < 0.01 versus normal serum, P < 0.002 versus LB serum.
^{g}P < 0.02 versus normal serum, P < 0.001 versus LB serum.
^{h}P < 0.02 versus normal serum, P < 0.002 versus LB serum.
ⁱP < 0.01 versus normal serum, P < 0.02 versus LB serum.
^{j}P < 0.001 versus normal serum, P < 0.001 versus LB serum.
^{k}P < 0.001 versus normal serum, P < 0.001 versus LB serum.
^{l}P < 0.001 versus normal serum, P: not significant versus LB serum.
^{m}P < 0.001 versus normal serum, P: not significant versus LB serum.

### Example 2

### Isolation and Characterization of the Serum Factor Associated with CR

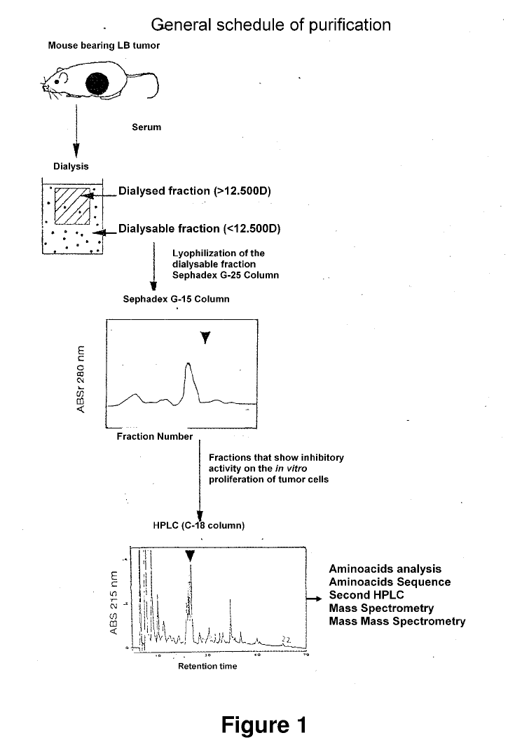

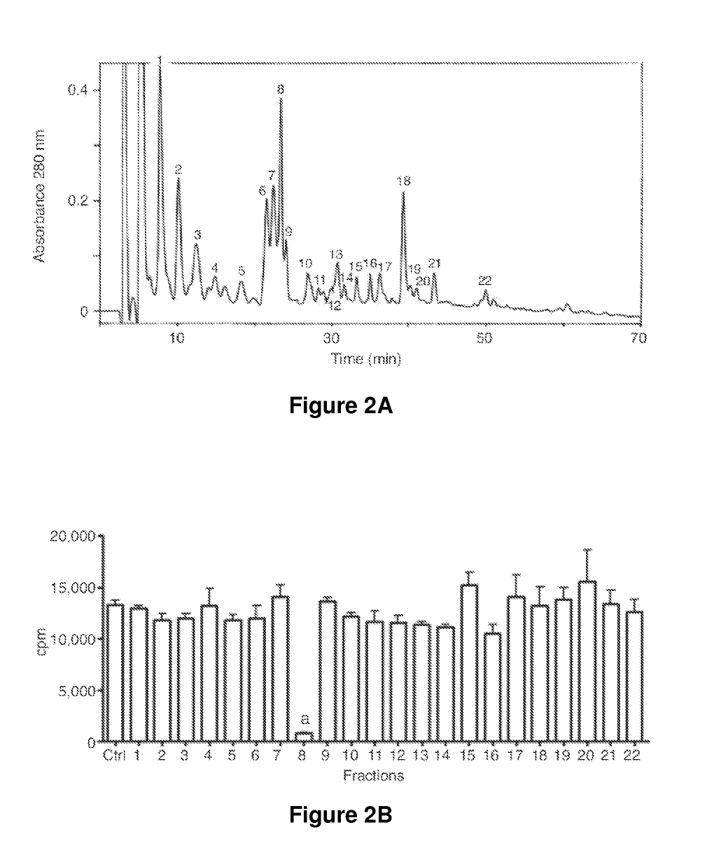

Fractionation of serum from mice bearing a subcutaneous LB tumor (size≧2,000 mm³) was carried out through several steps of purification (FIG. 1). Serum from normal mice was similarly fractioned as control. At all stages of the purification, the presence of the inhibitory factor was monitored by the [³H]-thymidine uptake assay as a measure of LB tumor cell proliferation. First, serum was decomplemented and subjected to dialysis. The inhibitory factor was recovered only in the dialyzable fraction (MW<12,500 Da) and was then concentrated by lyophilization and applied successively to Sephadex G-25 and G-15 chromatographic columns, where activity was recovered at fractions corresponding to a molecular weight below 1,000 Da. These fractions were lyophilized and further purified with an HPLC column C18 in a gradient of acetonitrile in TFA (first HPLC). In 12 similar and independent experiments, growth inhibitory activity was systematically recovered in only 1 fraction eluted at near 20% acetonitrile (FIG. 2).

In order to evaluate the presence or absence of fatty acids and other lipids in the active fraction from the first HPLC, a lipid analysis was carried out: from total lipid extracts, putative individual phospholipids, phosphoinositides, cholesterol and fatty acids were separated and quantified as previously reported by Márquez M G, Nieto F L, Fernández-Tome M, Favale N O, Sterin-Speziale N. Membrane lipid composition plays a central role in the maintenance of ephitelial cell adhesion to the extracellular matrix. Lipid 2008; 43: 343-352. No measurable amounts of phospholipids, phosphoinositides, cholesterol or fatty acids were detectable. In addition, the putative presence of urea, creatinin, uric acid and polyamines was evaluated as previously reported (Ruggiero et al. 1990, op. cit. and Iversen et al., op. cit.). Prostaglandins A1, A2 and J were quantified by HPLC by comparing the retention time of the sample with commercial standards. Prostaglandin E2 was evaluated using an enzyme immunoassay kit (Cayman Chemical, Ann Arbor; MI).

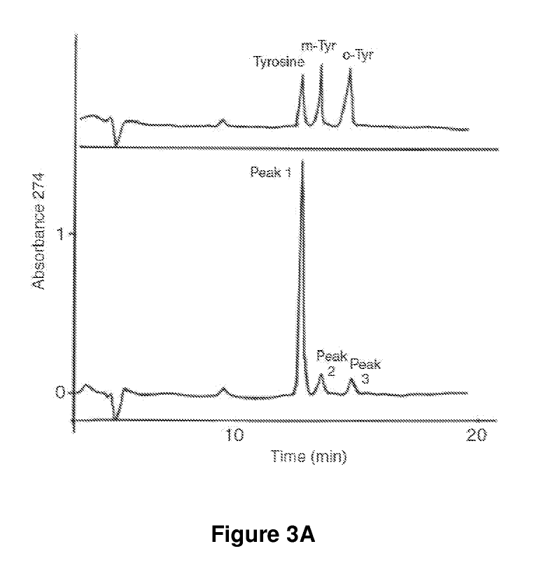

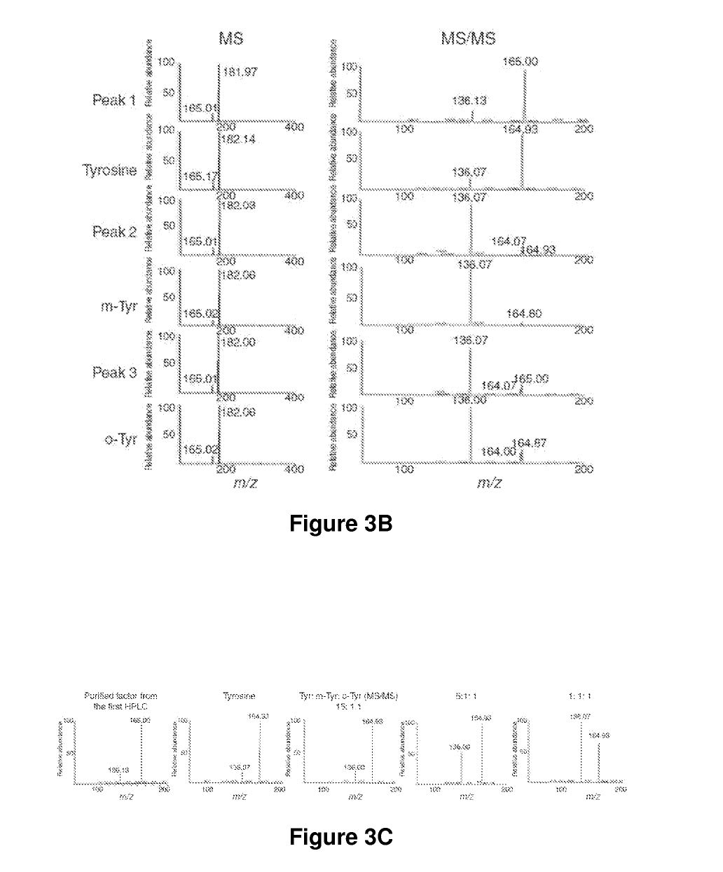

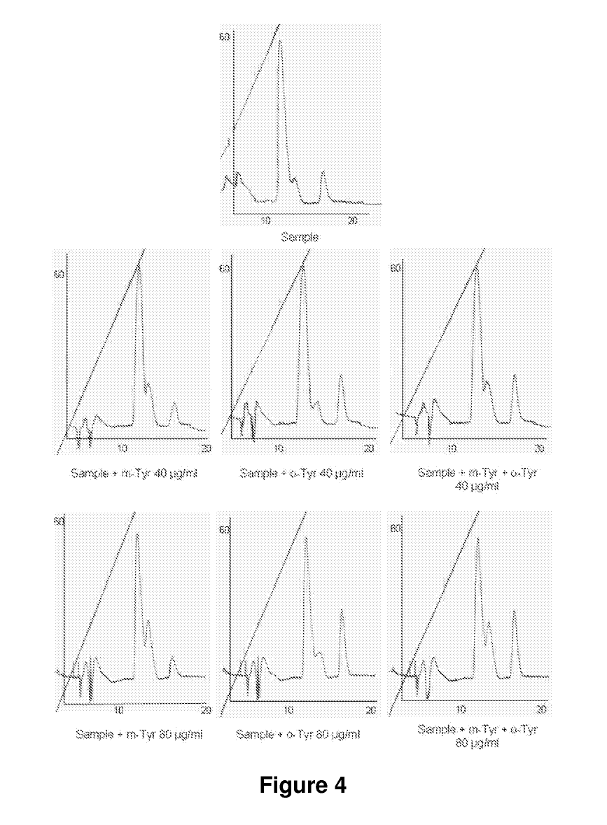

At first approach, characterization of this active fraction only revealed the presence of tyrosine not incorporated into a peptide (as evaluated by amino acid analysis and sequencing and by MS and MS/MS spectrometry) but not of putative inhibitory factors of low molecular weight sometimes present in biological fluids such as fatty acids, polyamines, creatinin, uric acid, urea, and prostaglandins E2, A1, A2, and J. This result was puzzling because tyrosine is neither inhibitory on tumor cell proliferation nor a common product of MDSC, or the result of an oxidative damage as the serum factor seemed to be. The elucidation of this puzzle began when this active fraction was applied to a second HPLC, in which the gradient was methanol in TFA, and yielded 3 peaks—instead of only 1 obtained in the first HPLC (FIG. 3A). The first and more conspicuous peak was characterized as tyrosine by comparing its retention time in the gradient with that of commercial tyrosine and by MS and MS/MS spectrometry. The second and the third peaks were characterized as 3-hydroxyphenylalanine (commonly known as meta-tyrosine or m-tyrosine) and 2-hydroxyphenylalanine (ortho-tyrosine or o-tyrosine), respectively, 2 isomers of tyrosine that are thought to be absent from normal proteins. It is worth noting that tyrosine and its isomers share the same MS spectrum (a major signal at m/z 182, consistent with a protonated molecule) but they can be distinguished by the relative abundance of the ions resulting from the fragmentation of the protonated molecule by the MS/MS analysis (FIG. 3B). To further confirm the identity of these isomers, graded concentrations of m-tyrosine and o-tyrosine were added to the biological sample, resulting in an increase of the intensity of the peaks 2 and 3, respectively, in a dose-dependent manner (FIG. 4). That is, the active fraction was actually a mixture composed by tyrosine:m-tyrosine:o-tyrosine in a proportion near 19:1.4:1 (tyrosine=600±134 μg/mL, m-tyrosine=44±11 μg/mL, and o-tyrosine 31±4 μg/mL; mean of 3 experiments) as calculated by comparing the absorption at 274 nm of the 3 real peaks against calibration curves obtained from known concentrations of commercial tyrosine, m-tyrosine, and o-tyrosine. In addition, we could show that when the ratio between the amount of tyrosine and that of the sum of its isomers was 7.5 or higher, such as it occurred in the mixture, MS/MS spectrometry was indistinguishable from that of tyrosine alone (FIG. 3C), explaining why the active fraction from the first HPLC seemed to contain tyrosine only.

### Example 3

### In Vitro Evaluation of the Inhibitory Activity of m-Tyrosine and o-Tyrosine

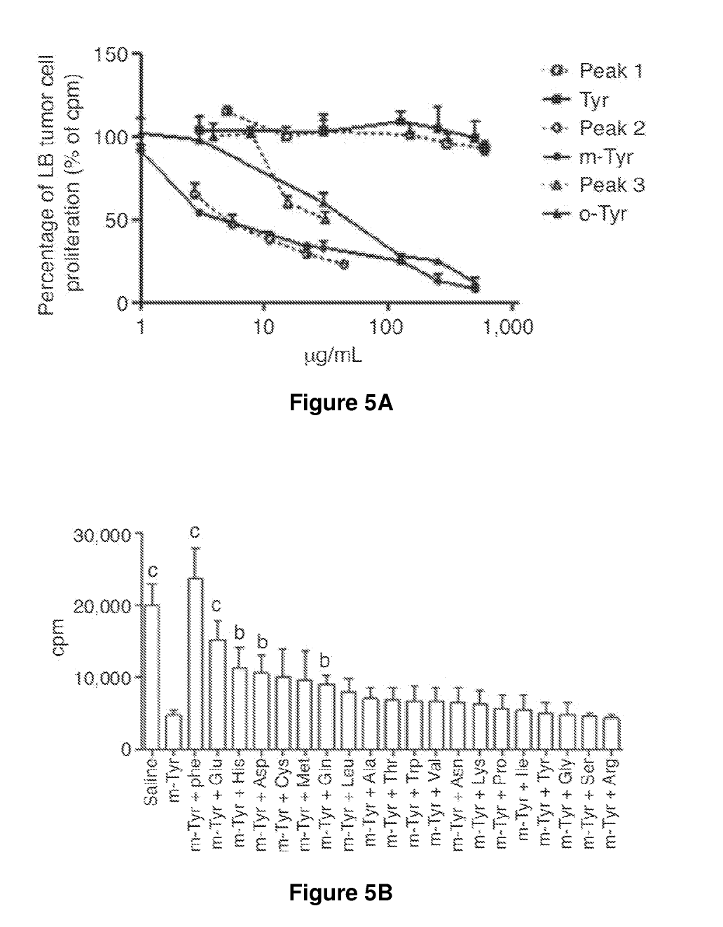

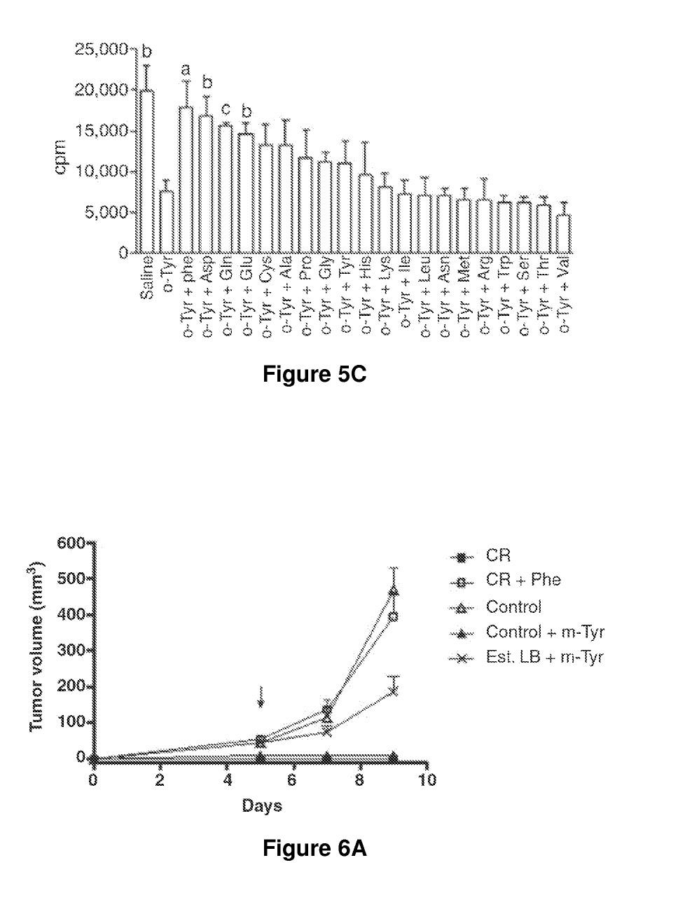

By using an array of equivalent concentrations of the serum purified peaks 2 and 3 from the second HPLC, and commercial m- and o-tyrosine, very similar dose-response curves on the in vitro LB tumor cell proliferation were obtained, indicating that m- and o-tyrosine could account for most of the growth-inhibitory activity present in the serum. The inhibitory effect produced by m-tyrosine was about 10 times more robust than that produced by o-tyrosine (GIU₅₀/mL for m-tyrosine=4.5±0.9 μg/mL; for o-tyrosine=46.7±5.2 μg/mL, P<0.002, n=3 experiments; FIG. 5A). This figure also shows that the peak 1 from the second HPLC and commercial tyrosine were innocuous for LB cells even with high concentrations of both.

Even an excess of commercial tyrosine in relation to m- and o-tyrosine—such as present in the active serum fraction—did not reduce the inhibitory activity of the latter. In contrast, phenylalanine and, to a lesser degree, glutamic acid, aspartic acid, and glutamine, counteracted the inhibitory effect produced by both m- and o-tyrosine in a dose-dependent manner whereas histidine only counteracted the inhibitory effect produced by m-tyrosine. No counteracting effect was observed with the remaining protein amino acids (FIGS. 5B and 5C).

The inhibitory effect produced by m- and o-tyrosine was reversible: after 18 hours of culture, LB tumor cells could reassume their normal growth by replacing the old medium (containing m- or o-tyrosine) by fresh medium (not shown).

The inhibitory effect of m- and o-tyrosine was not restricted to LB tumor cells: in vitro proliferation of MC-C, CEI, and C7HI tumor cells was also inhibited by m- and o-tyrosine in a dosedependent manner (not shown).

### Example 4

### In Vivo Evaluation of the Inhibitory Activity of m-Tyrosine and o-Tyrosine on LB Tumor Implants

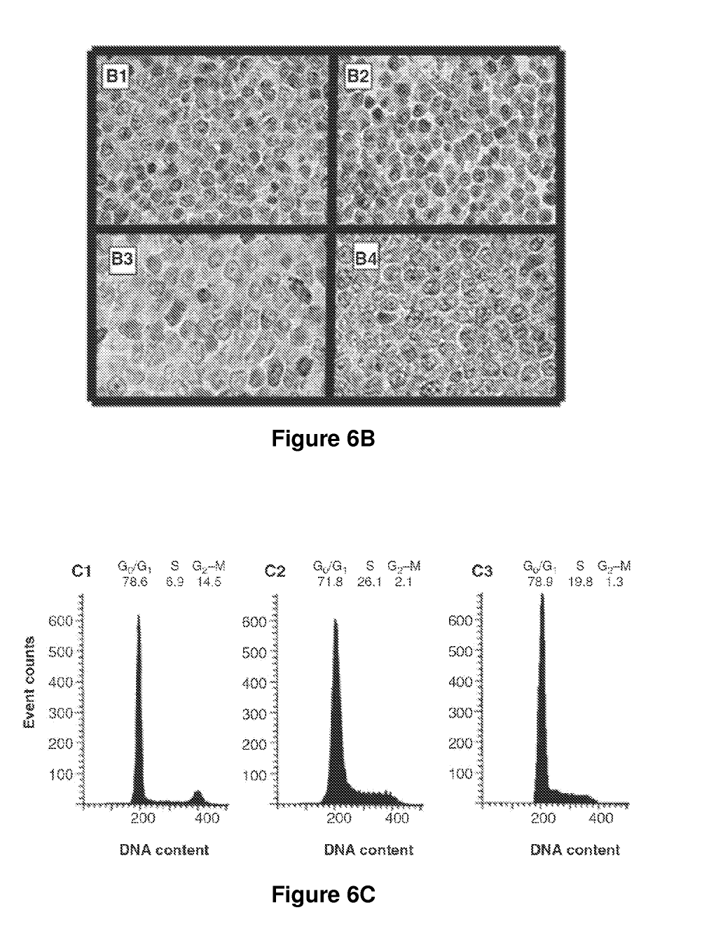

When phenylalanine was periodically inoculated at the site of a secondary LB tumor implant—otherwise inhibited by CR (Ruggiero et al. 1985, 1990, op. cit.; Bruzzo et al. 2010., op. cit.)—this secondary implant grew similarly to controls. On the contrary, when m-tyrosine was inoculated at the site of a primary tumor implant or systemically, this implant did not grow (FIG. 6A). o-Tyrosine was also inhibitory on LB tumor implants although its effect was weaker than that of m-tyrosine (not shown). In control tumors and in secondary tumor implants treated with phenylalanine, abundant tumor cells, displaying high expression of the cell proliferation marker Ki-67 protein (present in G₁-M phases but not in G₀), were observed (FIG. 6B). Reciprocally, the inhibition produced by exogenous injection of m-tyrosine mimicked the secondary tumor inhibition produced by CR: in both cases, tumor inhibition was associated with the presence of few tumor cells exhibiting low expression of Ki-67—meaning that most inhibited tumor cells were in G₀ (FIG. 6B), a decrease in G₂-M phases and an increase of the S phase population—considered the consequence of an S phase arrest (FIG. 6C).

In addition, both a secondary tumor inhibited by CR and a tumor inhibited by exogenous injection of m-tyrosine, could reassume their growth when transplanted in a normal mouse or when treatment with m-tyrosine was interrupted, respectively.

m-Tyrosine not only proved to be inhibitory on tumor implants but also on established s.c. LB tumors (FIG. 6A) and on ascitic LB tumor cells (not shown). Identical inhibition of tumor cells by m- and o-tyrosine was obtained in euthymic and in nude mice indicating that their inhibitory effects were not T-cell mediated.

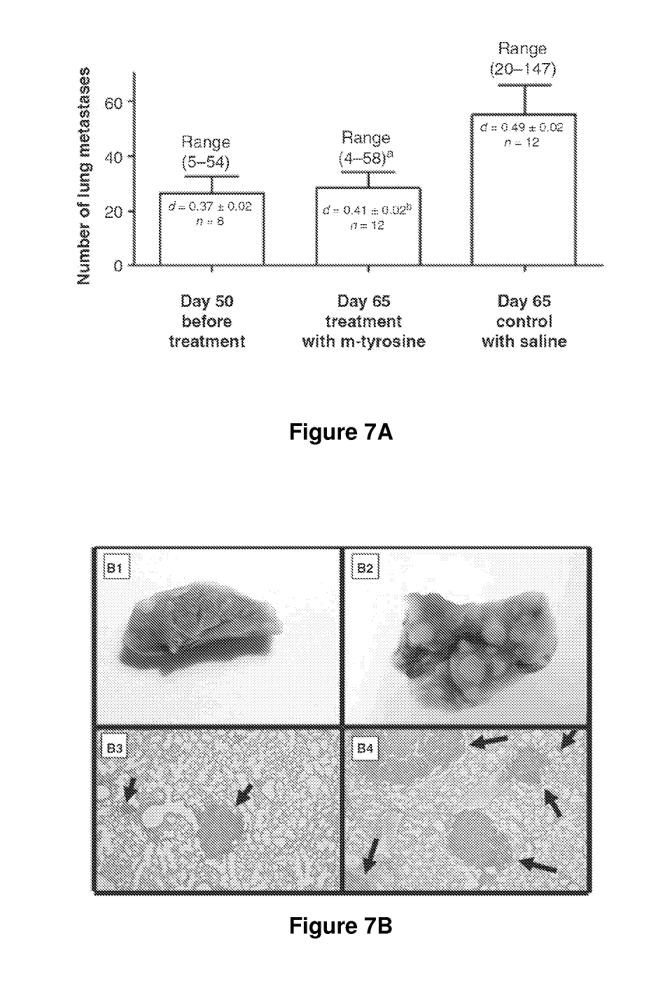

More compelling evidence supporting the contention that m- and o-tyrosine cause (at least in part) the second peak of CR was provided by the following experiment: LB primary tumor—bearing mice bearing a secondary LB tumor implant—inhibited by CR—were treated with gemcitabine plus catalase for 4 consecutive days. As expected by the results shown in Table 1, the titer of serum antitumor activity dropped to control values 2 days after the first inoculation while the previously arrested secondary tumor began to grow. HPLC analysis did not reveal the presence of m- and o-tyrosine in the serum lacking antitumor activity; in contrast, in nontreated tumor-bearing mice, where the secondary tumor was permanently inhibited, the serum displayed both a high titer of antitumor activity and the presence of m- and o-tyrosine. Furthermore, m-tyrosine also showed sharp inhibitory effects—without exhibiting toxic side effects—on the growth of MC-C fibrosarcoma and CEI epidermoid carcinoma—2 tumors that induce CR—and on the growth of established spontaneous lung metastases generated by the highly metastatic C7HI mammary adenocarcinoma that does not induce CR but is sensitive to the CR induced by other tumors (FIG. 7).

MC-C Tumor:

Twelve mice received a s.c. implant of 5×10⁶ MC-C tumor cells in the right flank and 25 days later they received a secondary implant of 2×10⁶ MC-C tumor cells in the left flank. Six (out of 12) mice did not receive any additional treatment and, as expected by previous experiments (quotations 13 and 20 of the manuscript) the secondary implant was strongly inhibited (tumor volume 20 days after the implant in the left flank, that is at day 45 of primary tumor growth, was =194±52 mm³). The remaining 6 mice received 0.2 ml of phenylalanine (500 μg/ml) at the site of the secondary implant, daily, for 19 days, starting 1 hour later than the secondary implant. These secondary implants grew as in 6 controls only receiving the implant in the left flank (tumor volumes of secondary tumor+phenylalanine and of controls, 20 days after the implant in the left flank were =680±152 mm³ and 524±88 mm³, respectively). On the other hand, when control mice that only had received the tumor implant in the left flank, also received m-tyrosine (0.2 ml of 500 μg/ml) daily for 19 days at the site of the tumor implant (n=6), this implant was significantly inhibited (tumor volume 20 days after the implant in the left flank was =212±57 mm³) mimicking the inhibitory effect produced by the presence of a primary MC-C tumor in the right flank. Significance: Secondary tumor vs. control: p<0.01; vs. secondary tumor+phenylalanine: p<0.02. Control+m-tyrosine vs. control and vs. secondary tumor+phenylalanine: p<0.02.

CEI Tumor:

Twelve mice received a s.c. implant of 1×10⁶ CEI tumor cells in the right flank and 28 days later they received a secondary implant of 3×10⁵ CEI tumor cells in the left flank. Six (out of 12) mice did not receive any additional treatment and, as expected by previous experiments (quotations 9 and 22 of the manuscript) the secondary implant did not grow (tumor volume 20 days after the implant in the left flank, that is at day 48 of primary tumor growth, was =0 mm³). The remaining 6 mice received 0.2 ml of phenylalanine (500 μg/ml) at the site of the secondary implant, daily, for 19 days, starting 1 hour later than the secondary implant. These secondary implants grew as in 6 controls only receiving the implant in the left flank (tumor volumes of secondary tumor+phenylalanine and of controls, 20 days after the implant in the left flank were 358±38 mm³ and 373±93 mm³, respectively). On the other hand, when control mice that only had received the tumor implant in the left flank, also received m-tyrosine (0.2 ml of 500 μg/ml) daily for 19 days at the site of the tumor implant (n=6), this implant did not grow (tumor volume 20 days after the implant in the left flank was =0 mm³) mimicking the inhibitory effect produced by the presence of a primary CEI tumor in the right flank. Significance: Secondary tumor and control+m-tyrosine vs. control: p<0.01; vs. secondary tumor+phenylalanine: p<0.001.

O-tyrosine was also inhibitory on CEI and MC-C tumor implants although its effect was weaker than that of m-tyrosine (not shown).

Both series of experiments together with the experiments carried out with LB tumor, strongly suggested that m- and o-tyrosine would be responsible for the second peak of CR induced by LB, MC-C and CEI tumors, raising the possibility that this mechanism can be generalized to explain the phenomenon of CR in other tumor models.

### Example 5

### In Vivo Evaluation of the Response of Primary Vs. Secondary Tumors to the Tyrosine Isomers

In an attempt to understand why a secondary tumor is inhibited while the primary one continues to grow, mice bearing an s.c. LB-growing tumor, received an implant of a sterile polystyrene sponge fragment (about 0.5 cm³) near the site of tumor growth and a similar sponge fragment at the contralateral flank, at the site of a putative secondary tumor implant. A sponge implanted in the flank of normal mice, served as control. Seven days later, the cell-free fluids collected from the sponges were dialyzed and amino acid content of the dialyzable fraction was evaluated. The fluid from the sponges placed near the primary tumor (size≧2,000 mm³) showed a significantly higher concentration of 16 (out of 20) amino acids than that observed in the sponges implanted at the contralateral flank, including the 5 amino acids (phenylalanine, glutamic acid, aspartic acid, glutamine, and histidine) that counteracted the inhibitory effect of m- and/or o-tyrosine. This could protect, at least in part, the primary tumor against the antitumor effects mediated by m- and o-tyrosine. In fact, the fluid collected from the sponges placed near the primary tumor exhibited an inhibitory effect on in vitro tumor cell proliferation (170±25 GIU₅₀/mL, n=4 experiments) more than twice lower than the fluid collected from the sponges placed at the contralateral flank (349±67 GIU₅₀/mL; n=4; P<0.05) and than the serum from LB tumor—bearing mice (380±25 GIU₅₀/mL; n=4; P<0.002). The basal titer of both the fluid collected from control sponges and normal serum—without detectable amounts of m- and o-tyrosine—were 65±25 GIU₅₀/mL (n=4) and 47±10 GIU₅₀/mL (n=4), respectively.

### Example 6

### In Vivo Evaluation of the Effect of Amino Acids from Primary Vs. Secondary Tumor on the Inhibitory Action of m-Tyrosine and o-Tyrosine

To test directly whether a “cocktail” of amino acids similar to that detected close to the “primary site” was more counteracting of the antitumor effects produced by m- and o-tyrosine than a “secondary site cocktail,” different concentrations of a mixture of m-tyrosine and o-tyrosine (ratio 1:1) were assayed on LB tumor cell proliferation, alone or together with a primary or a secondary site cocktail of amino acids. The tumor growth inhibitory effect produced by the mixture (GIU₅₀/mL=18±3 μg/mL) was counteracted by both the primary and the secondary sites cocktails. However, the counteracting effect of the primary site cocktail was more than twice greater than that of the secondary site cocktail (GIU₅₀/mL of m-tyrosine and o-tyrosine in the presence of the primary site cocktail was =170±8 μg/mL vs. 70±4 μg/mL in the presence of the secondary site cocktail, P<0.01, mean of 2 experiments) indicating that the inhibitory effect generated by m- and o-tyrosine could be tempered near the primary tumor as compared with the secondary tumor site.

### Example 7

### Inhibitory Action of m-Tyrosine and o-Tyrosine at the Molecular Level

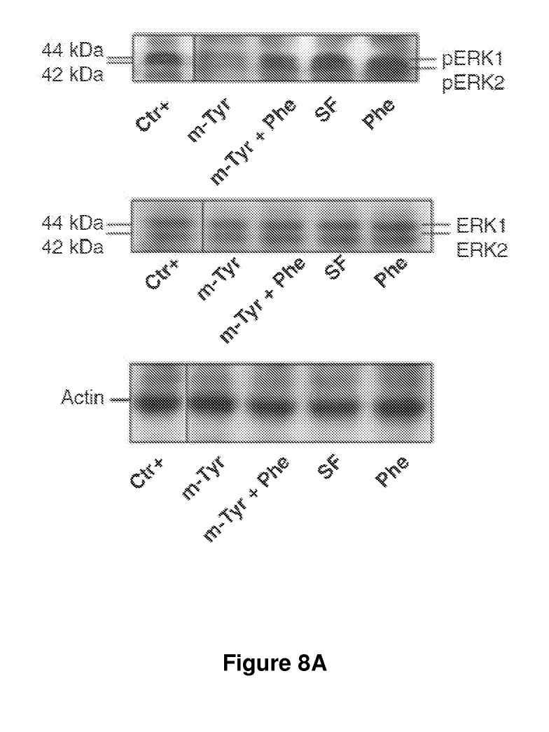

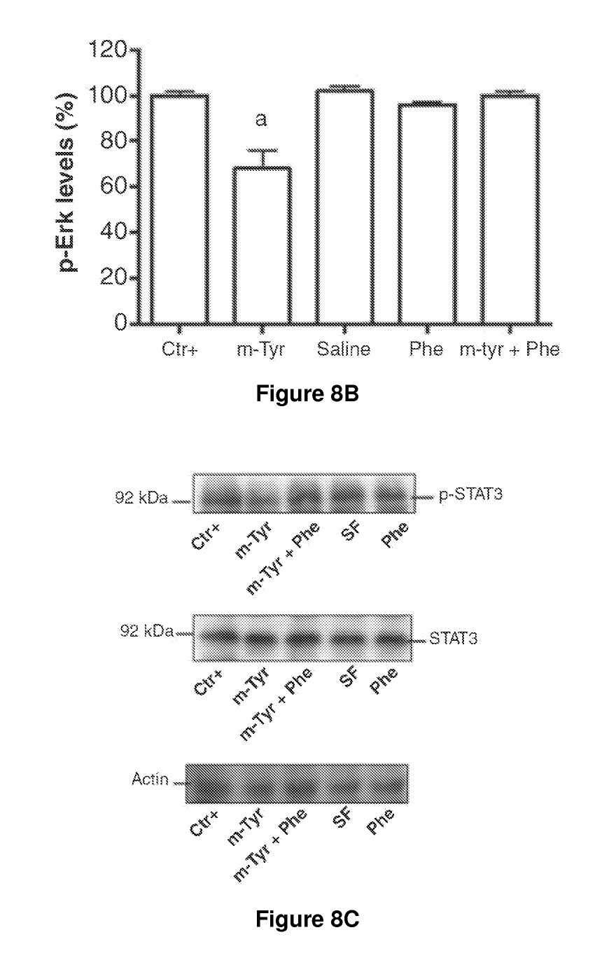

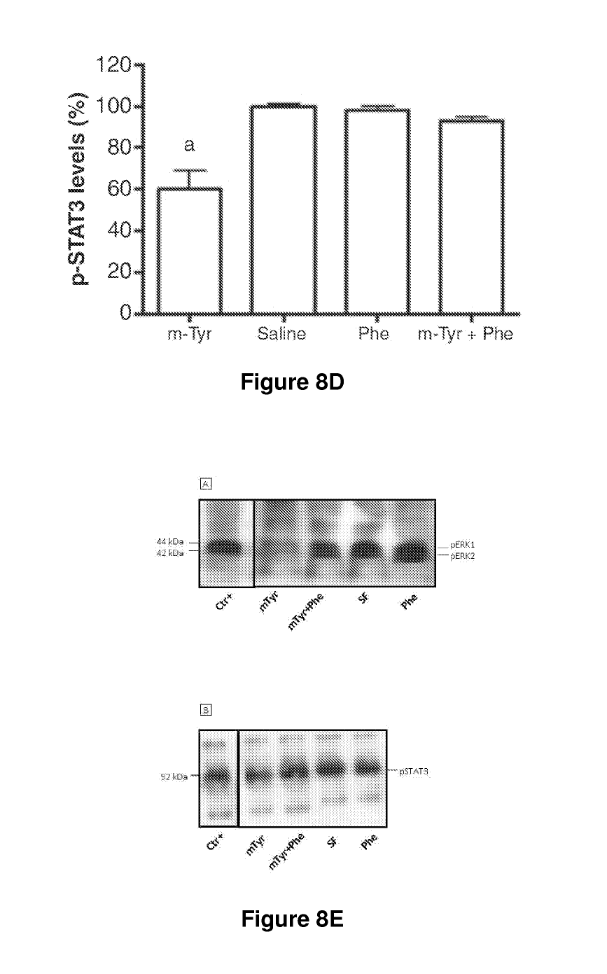

LB tumor cells cultured with m-tyrosine displayed significant changes in the pattern of protein phosphorylation in a dose-dependent manner (not shown). On this basis, we analyzed, first, the effect of m-tyrosine on ERK1 and ERK2 as 2 examples of protein kinases that are activated by the mitogen activated protein/extracellular signal-regulated kinase (MAP/ERK) signal transduction pathway that normally couples intracellular responses associated with cell proliferation, to the binding of growth factors to cell surface receptors (Kohno M, and Pouyssegur J. 2006. Targeting the ERK signaling pathway in cancer therapy. Ann Med; 38:200-211). Both, ERK1 and ERK2 are constitutively activated in LB tumor cells, but when these cells were cultured with m-tyrosine, that activation was significantly reduced as early as 3 minutes after the onset of the culture, whereas the addition of phenylalanine reversed that effect (FIGS. 8A and 8B). Second, we analyzed the effect of m-tyrosine on STAT3, which is activated (among others) by MAP/ERK cascade and in turn activates several genes involved in cell-cycle progression (Lassmann S, Schuster I, Walch A, Göbel H, Jütting U, Makowiec F, et al. 2007. STAT3mRNA and protein expression in colorectal cancer: effects on STAT3-inducible targets linked to cell survival and proliferation. J Clin Pathol 60:173-179; Aznar S, Valeron P F, del Rincon S V, Perez L F, Perona L F, and Lacal J C. 2001. Simultaneous tyrosine and serine phosphorylation of STAT3 transcription factor is involved in Rho A GTPase oncogenic transformation. Mol Biol Cell 12:3282-2394). STAT3 is also constitutively activated in LB tumor cells, but when these cells were cultured with m-tyrosine, that activation was significantly reduced 8 hours after the onset of the culture, whereas the addition of phenylalanine reversed that effect (FIGS. 8C and 8D). The low expression of p-STAT3 was temporally correlated to a low expression of Ki-67 protein, to a cell-cycle distribution identical to that observed in tumor cells inhibited by CR or by m-tyrosine in vivo and to the onset of the inhibition of [³H]-thymidine uptake by LB tumor cells (not shown). Furthermore, with different concentrations of m-tyrosine, we could show that the inhibitory effect produced by m-tyrosine on LB tumor cell proliferation was proportional to the reduction of the expression of p-ERK 1/2, p-STAT3, and Ki-67 protein. In consequence, the above experiments suggest that the partial inactivation of ERK1/2 and STAT3 mediated by m-tyrosine could be involved—at least in part—in a relatively rapid mechanism that may drive tumor cells into a state of dormancy.

### Example 8

### In Vivo Evaluation of the Inhibitory Activity of m-Tyrosine and o-Tyrosine on C7HI Tumors

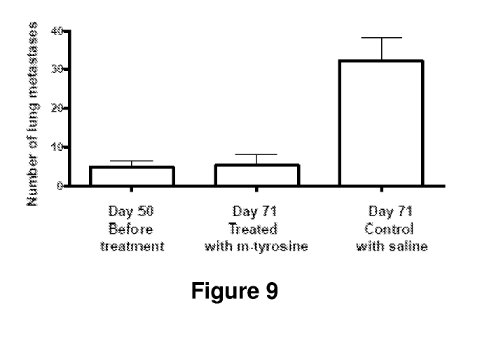

C7HI is a breast carcinoma that generates a high number of pulmonary and hepatic metastases, the number of pulmonary metastases being higher than the number of hepatic metastases. In this experiment, 36 mice received one implant each of 5×10⁵ tumor C7HI cells. 50 days after implanting the tumor cells, 12 mice were sacrificed for evaluating the number and size of pulmonary and hepatic metastases. The remaining 24 mice were divided in two groups of 12 mice each. The experimental group received 1.0 mg of meta-tyrosine diluted in 0.1 ml of saline per mouse per day. Different administration routes were tried and endovenous administration was found to be the better. The second group (control) received in 0.1 ml of saline per mouse per day. After 21 days of treatment, all mice were sacrificed and the number and size of the metastases were evaluated. As shown in FIG. 9, treatment with meta-tyrosine prevented the growth of established macroscopic pulmonary metastases. Furthermore, it also prevented growth of microscopic metastases. This inhibitory activity was reflected not only in that the number of metastases of both groups was the same, and in some cases lower, as compared with the number of metastases at the beginning of the treatment (as estimated by the data produced by the 12 mice sacrificed before starting the treatments), but also in that their size was either the same or smaller. This is remarkable because of the 12 experimental mice, there was one showing a low response to the treatment. Should the values measured in this particular mouse be excluded from the pool, the number of metastases at day 71 in the experimental group would have been less than half the number of metastases observed before the start of the treatment at day 50.

Regarding hepatic metastases, at day 71 the mice in the control group presented hepatic metastases, while no metastases were detected in the liver of the mice treated with meta-tyrosine.

The experiment was repeated using doses of 0.5 mg and 0.1 mg of meta-tyrosine per mouse per day, with similar results.

Finally, the experiment was repeated but using orto-tyrosine instead of meta-tyrosine. Treatment with orto-tyrosine also showed an inhibitory effect, although to a lesser extent than treatment with meta-tyrosine, which correlates with the lesser antitumoral in vitro activity shown in previous examples.

### Example 9

### Estimation of Therapeutically Effective Dose in Humans

Data of example 8 show that the treatment by endovenous route for three weeks inhibited the growth of established metastases in lung and liver. With the aim of extrapolating these data and doses to human beings, it can be calculated that since a mouse weights on average 30 grams, doses of 1 mg/mouse, 0.5 mg/mouse and 0.1 mg/mouse represent doses of 33.3, 16.7 and 3.3 mg/kg of body weight, respectively. Assuming that an adult human being weights on average 70 kg, the meta-tyrosine dose with anti-metastatic effect that a human patient should receive is 2.3, 1.2 or 0.23 grams per day. That is, the minimal dose with proven anti-metastatic activity would be of 230 mg per day.

### Example 10

### Evaluation of Toxicity of the Tyrosine Isomers in Humans

The effect of meta-tyrosine and ortho-tyrosine on the immune response triggered by a conventional antigen was evaluated. Ram red cells (RRC) were used. Results showed that administering the higher dose of the isomers does not affect the anti-RRC antibody levels either in tumor bearing mice or in normal mice. This higher dose of tyrosine isomers did not affect the hematologic populations of the blood or in the spleen either.

The higher dose of both meta-tyrosine and ortho-tyrosine does not seem to produce severe damage since no histological alterations were evident in any of several analyzed tissues, and mice remained in good health after three weeks of treatment.

1-18. (canceled)

19. A method for preventing or treating cancer, wherein an isomer of tyrosine selected from meta- and/or ortho-tyrosine is administered in a therapeutically effective amount to a subject in need thereof.

20. The method of claim 19, wherein the cancer is selected from the group consisting of leukemia (including LB leukemia), fibrosarcoma (including MC-C), primary melanomas, carcinomas (including CEI), and breast, testicular, ovarian, lung, colorectal, and bladder cancers.

21. The method of claim 19, wherein it is for preventing or reducing the growth of a primary tumor.

22. The method of claim 19, wherein it is for preventing or reducing the growth of cancer metastases after the removal of a primary tumor, or after other surgical injuries or stressors that may promote the escape of metastases from dormancy.

23. The method of claim 22, wherein the cancer is known to show sudden acceleration of metastases after surgical removal of a tumor.

24. The method of claim 23, wherein the cancer is selected from the group comprising primary melanomas and breast, testicular, ovarian, lung, colorectal, and bladder cancers.

25. The method of claim 19, wherein the isomer of tyrosine is meta-tyrosine.

26. The method of claim 19, wherein the isomer of tyrosine is administered intravenously.

27. The method of claim 19 wherein the isomer of tyrosine is injected at or near the site of the cancer cells whose growth prevention or reduction is sought.

28. The method of claim 19 wherein the therapeutically effective amount is between 33.3 mg/kg of body weight and 3.3 mg/kg of body weight.

29. The method of claim 19 wherein the therapeutically effective amount is 3.3 mg/kg of body weight and the tyrosine isomer is meta-tyrosine.

30. A pharmaceutical composition which comprises an isomer of tyrosine selected from the group consisting of meta- and/or ortho-tyrosine, preferably meta-tyrosine, and a pharmaceutically acceptable carrier.

31. The pharmaceutical composition of claim 30, wherein the pharmaceutical composition is for the prevention or treatment of cancer.
